(19) United States
(12) Patent Application Publication
(10) Pub. No.: US 2015/0173944 A1
(43) Pub. Date: Jun. 25, 2015

(54) SYSTEMS AND METHODS FOR ATTACHING A SURGICAL INSTRUMENT TIP

(71) Applicant: NOVARTIS AG, Basel (CH)

(72) Inventors: Thomas Linsi, Schaffhausen (CH); Guido Vezzu, Pfungen (CH)

(21) Appl. No.: 14/576,398

(22) Filed: Dec. 19, 2014

Related U.S. Application Data
(60) Provisional application No. 61/919,866, filed on Dec. 23, 2013.

(51) Int. Cl. A61F 9/007; A61B 17/29; A61B 17/30

(52) U.S. Cl. CPC A61F 9/007; A61B 17/30; A61B 17/2909; A61B 2017/0046

(57) ABSTRACT

A surgical instrument for treating a medical condition of a patient includes a protruding tool and a body assembly carrying the protruding tool. The body assembly includes a plurality of flexible claws and a locking ring moveable along the flexible claws between a locked position and an unlocked position. A main body forms a hand grip portion and includes an engagement interface portion configured to cooperate with the flexible claws to secure the body assembly to the main body when the locking ring is in the locked position.

### PRIORITY INFORMATION

The present application claims the benefit of U.S. Provisional Patent Application No. 61/919,866, filed Dec. 23, 2013 the disclosure of which is incorporated by reference herein in its entirety.

### BACKGROUND

The devices, systems, and methods disclosed herein relate generally to surgical instruments, and more particularly, to surgical instruments for treating an ocular condition.

Some single-use surgical instruments include a detachable surgical tip that may include or may carry a surgical tool. The tool may be used to directly interface with and invasively or non-invasively treat a patient. After the surgical instrument has been used, the surgeon or assistant may remove the tip, along with the tool, from the remaining portion of the instrument and discard it. Additional portions of the surgical instrument, such as a handpiece for example, may be sterilized and reused. Discarding only the surgical tip while reusing the rest of the handpiece may reduce costs to hospitals, while still maintaining a high level of safety and hygiene for the patient.

Some surgical instruments with detachable tips are configured with a non-symmetric connection interface. Because of this, axial loads on the surgical tip from the handpiece may result in non-symmetric loading on the surgical tip, and subsequently, on the surgical tool. This may result in pivoting or deflection of the tip and the tool. The pivot or deflection is compounded when the tool extends a relatively large distance from the rest of the surgical tip.

The present disclosure is directed to addressing one or more of the deficiencies in the prior art.

### SUMMARY

In an exemplary aspect, the present disclosure is directed to a surgical instrument for treating a medical condition of a patient. The surgical instrument may include a protruding tool configured to interface with and treat patient tissue and may include a body assembly carrying the protruding tool. The body assembly may include a plurality of flexible claws and a locking ring moveable along the flexible claws between a locked position and an unlocked position. A main body may form a hand grip portion to be grasped by a user during a medical procedure. The main body may include an engagement interface portion configured to cooperate with the flexible claws to secure the body assembly to the main body when the locking ring is in the locked position.

In an aspect, the body assembly comprises a tip body and each flexible claw of the plurality of flexible claws comprises a first portion attached to the tip body and a second portion opposite the first portion. The second portion may include a protruding locking element configured to engage the engagement interface portion. In an aspect, the protruding locking element comprises a shoulder surface that engages the engagement interface portion when the locking ring is in the locked position. In an aspect, the first portion has a first thickness and the second portion has a second thickness greater than the first thickness. The first portion may be arranged to elastically deflect under loading from the locking ring. In an aspect, each flexible claw of the plurality of flexible claws comprises an inner facing surface and an outer facing surface. The inner facing surface may include the protruding locking element, and the outer facing surface may include a slide stop that interfaces with the locking ring. In an aspect, the plurality of flexible claws is biased to flare radially outwardly when the locking ring is in the unlocked position. In an aspect, the flexible claws are symmetrically disposed in a radial configuration to provide symmetrical loading under an axial load. In an aspect, the flexible claws and the engagement interface portion are shaped in a manner to permit rotation of the body assembly about the engagement interface portion when the locking ring is in the locked condition. In an aspect, the body assembly carries a tool actuation mechanism. The tool actuation mechanism may be actuatable via axial movement of an actuation assembly in the main body. In an aspect, the engagement interface portion of the main body includes a radial groove that receives at least a portion of the flexible claws when the locking ring is in the locked position.

In another exemplary aspect, the present disclosure is directed to a surgical instrument for treating a medical condition where the surgical instrument includes a protruding, actuatable tool configured to interface with and treat patient tissue and includes a body assembly having a distal end and a proximal end and a longitudinal axis extending therebetween. The tool may project from the distal end along the longitudinal axis. The proximal end may comprise a plurality of flexible claws symmetrically disposed about the longitudinal axis. A locking element may be moveable axially between a locked position and an unlocked position. A main body forms a gripping portion to be grasped by a user during a medical procedure. The main body may include an axially aligned engagement interface portion configured to be engaged by the plurality of symmetrically disposed flexible claws so that axial loading on the engagement portion is countered by the flexible claws in a symmetrical manner.

In an exemplary aspect, the present disclosure is directed to a method of assembling a surgical instrument for treating a medical condition. The method may include orienting a locking element on surgical tip relative to a plurality of flexible claws on the surgical tip so that the flexible claws are in an unlocked position; introducing an engagement interface portion of a main body in an axial direction until the engagement interface portion is adjacent opposing flexible claws of the plurality of flexible claws, the engagement interface portion including a transverse lock surface; and advancing the locking element, relative to the plurality of flexible claws and relative to the engagement interface portion, from an unlocked state to a locked state so that the flexible claws move from the unlocked position to a locked position where a portion of each flexible claw of the plurality of flexible claws is disposed axially in-line with the transverse locking surface to mechanically prevent removal of the engagement interface portion from the surgical tip.

In an aspect, the method step of introducing an engagement interface portion comprises introducing the engagement interface portion between opposing flexible claws. In an aspect, the step of advancing the locking element from an unlocked state to a locked state comprises deflecting the flexible claws radially inwardly.

It is to be understood that both the foregoing general description and the following drawings and detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following.

### BRIEF DESCRIPTION OF THE DRAWINGS

The accompanying drawings illustrate embodiments of the devices and methods disclosed herein and together with the description, serve to explain the principles of the present disclosure.

FIG. 1 is an illustration of an elevation view of an exemplary surgical instrument according to an exemplary aspect of the present disclosure.

FIG. 2 is an illustration of an exemplary tip and a portion of a main body that may form a part of the surgical instrument of FIG. 1 according to an exemplary aspect of the present disclosure.

FIG. 3A is an illustration of a cross-sectional view of an exemplary tip connected with a part of a main body that may form a part of the surgical instrument of FIG. 1 according to an exemplary aspect of the present disclosure.

FIG. 3B is an illustration of a cross-sectional view of the exemplary tip connected with a part of the main body of FIG. 3A rotated 90 degrees according to an exemplary aspect of the present disclosure.

FIG. 4A is an illustration of a cross-sectional view of an exemplary tip without a protruding tool and in a locked position that may form a part of the surgical instrument of FIG. 1 according to an exemplary aspect of the present disclosure.

FIG. 4B is an illustration of a cross-sectional view of an exemplary tip without a protruding tool and in an unlocked position that may form a part of the surgical instrument of FIG. 1 according to an exemplary aspect of the present disclosure.

FIG. 5 is an illustration of a side view of an exemplary nose that may form a part of the tip of FIG. 2 according to an exemplary aspect of the present disclosure.

FIG. 6 is an illustration of a cross-sectional view of the exemplary nose of FIG. 5 according to an exemplary aspect of the present disclosure.

FIG. 7 is an illustration of an exemplary flexible claw that may form a part of the nose of FIG. 5 according to an exemplary aspect of the present disclosure.

FIG. 8 is an illustration of a side view of an exemplary locking ring that may form a part of the tip of FIG. 2 according to an exemplary aspect of the present disclosure.

FIG. 9 is an illustration of a cross-sectional view of the exemplary locking ring of FIG. 8 according to an exemplary aspect of the present disclosure.

FIG. 10 is an illustration of an exemplary slide mechanism that may form a part of the tip of FIG. 2 according to an exemplary aspect of the present disclosure.

FIG. 11 is an illustration of an attachment step for connecting a tip with a main body of a surgical instrument according to an exemplary aspect of the present disclosure.

FIG. 12 is a stylized illustration of an exemplary tip and main body showing symmetric force distribution according to an exemplary aspect of the present disclosure.

### DETAILED DESCRIPTION

For the purposes of promoting an understanding of the principles of the present disclosure, reference will now be made to the embodiments illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure relates generally to surgical instruments, systems, and methods that include a patient-interfacing tip removable from a main body, such as a handpiece. In at least some of the examples described herein, the tip may be easily attached to or removed from the handpiece. The tip and main body symmetrically connect so that axial loading on the tip is completely or nearly completely maintained in the axial direction. This symmetry inhibits or prevents lateral movement of the tip, or a tool carried by the tip, relative to the main body. This may help maintain the tip in axial alignment with the main body, and may result in a more consistently manufactured, more stable, and more predictable surgical instrument.

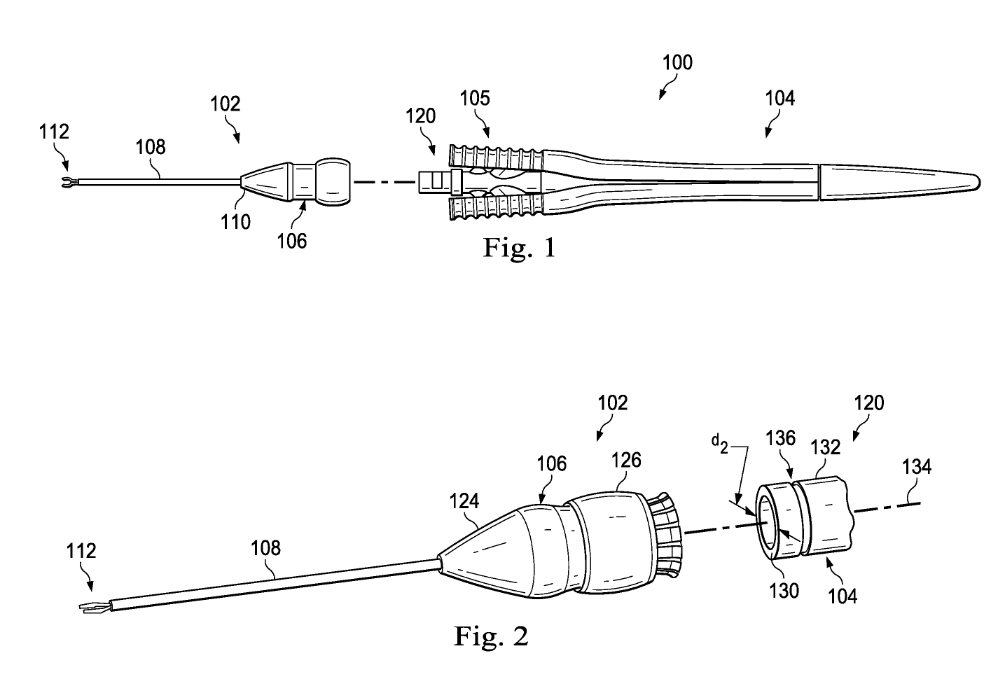

FIG. 1 shows an exemplary surgical instrument 100 according to an exemplary aspect of the present disclosure. In the exemplary embodiment of FIG. 1, the surgical instrument includes a tip 102 and a main body 104. In this embodiment, the surgical instrument 100 is forceps sized and arranged for treatment or evaluation of an ophthalmic condition in an eye of the patient. However, the principles of connection disclosed herein may be used with any surgical instrument having a separable tip and main body and may be used for any medical purpose, including those not related to ophthalmic conditions.

The tip 102 is selectively separable from the main body 104, as shown in FIG. 1, and may be connected to the main body 104 in a manner allowing the tip 102 to be controlled, actuated, or simply manipulated from the main body 104. In this example, as can be seen, the tip 102 includes a body assembly 106 and a distally protruding tool 108. The distally protruding tool 108 extends from a distal tip 110 of the body assembly 106. The body assembly 106 is configured to carry the distally protruding tool 108 and is configured to interface with the main body 104. The body assembly 106 will be described in greater detail below. In the embodiment shown, the distally protruding tool 108 is configured and arranged to interface with the patient to perform an action on the patient to treat, assess, or otherwise interact directly with the patient tissue. In the exemplary embodiment shown, the distally protruding tool 108 is a forceps and includes a distal tip 112 forming forceps jaws. In this example, the distally protruding tool 108 is configured to treat or assess an ophthalmic condition and may be sized to penetrate a portion of the eye, such as the globe of an eye for example, to treat the ophthalmic condition. As such, the diameter of the distally protruding tool 108 may be in the range of about, for example, 17 gauge (1.15 mm) to about 30 gauge (0.255 mm) Other sized tools are contemplated, both larger and smaller. Further, although forceps are mentioned, the distally protruding tool 108 may be scissors, tweezers, pliers, probes, scoops, or other tools. In the embodiment shown, the distally protruding tool 108 is configured to engage or be actuated by an actuation assembly (not shown) that may be carried on the main body 104 that opens or closes the forceps.

The main body 104 is configured to receive and carry the tip 102. As will be described further below, the main body 104 includes an engagement interface portion 120 configured to secure the tip 102 onto the main body 104. The main body 104 forms a graspable handle that may be held and manipulated by a surgeon or other health care provider during a medical procedure. The engagement interface portion 120 may form a part of an actuation assembly 105 carried on the main body 104 that may actuate the tool 108. In some aspects, the actuation assembly 105 axially advances and retracts a portion that interfaces with the tip 102 to actuate the tool 108.

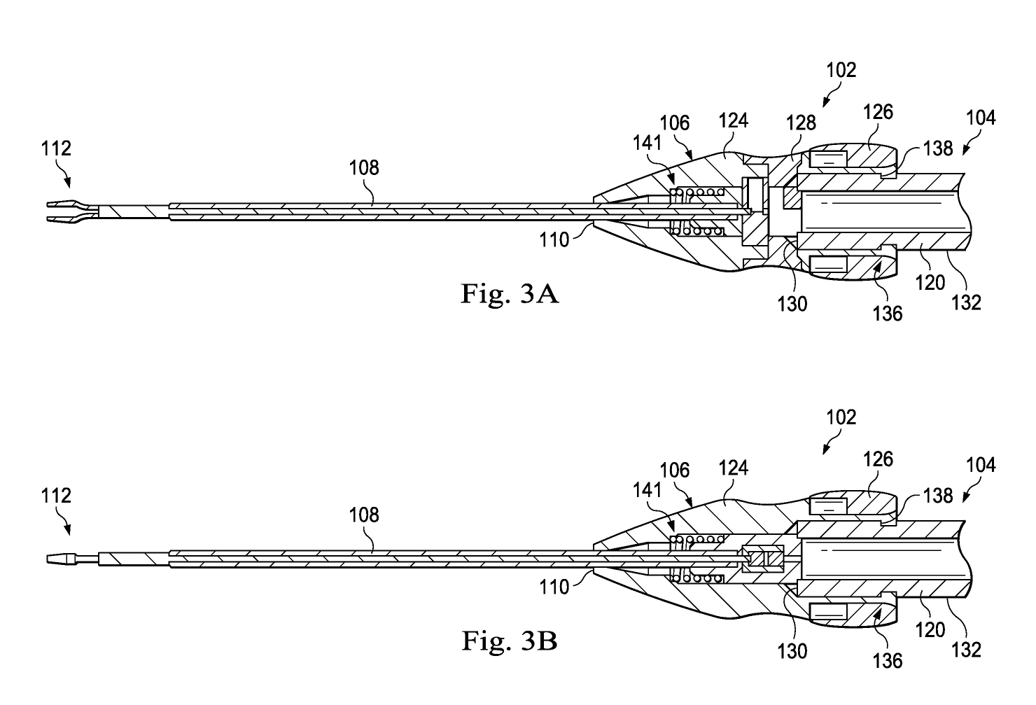

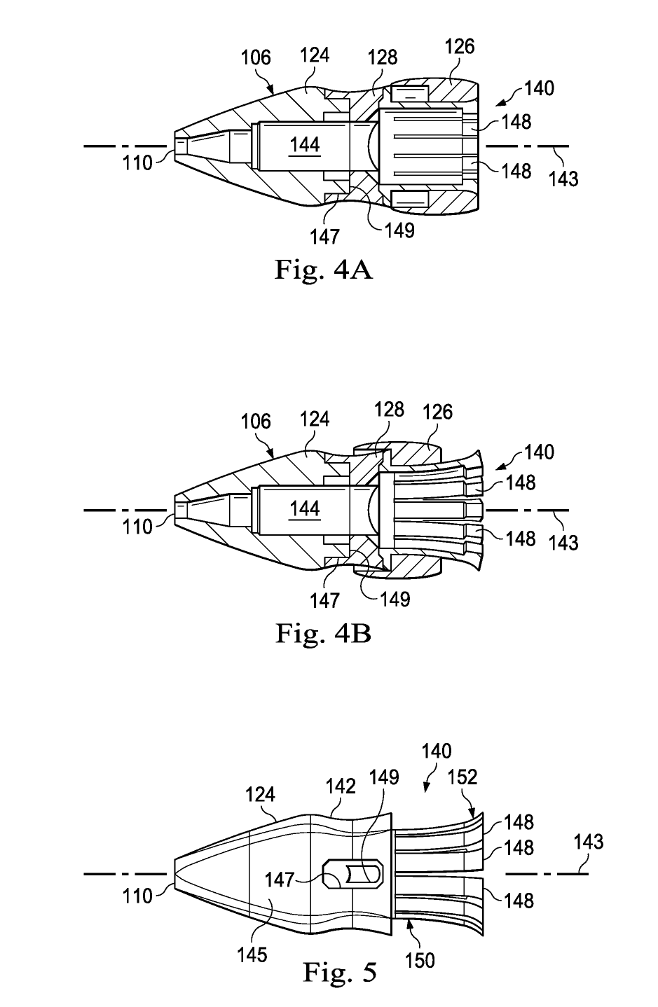

FIGS. 2, 3A, 3B, 4A, and 4B show the tip 102 or portions of the tip 102 assembled together. The engagement interface portion 120 of the main body 104 is also shown in FIGS. 2, 3A, and 3B. FIG. 2 shows a partially isometric view, while FIGS. 3A, 3B, 4A, and 4B show cross-sectional views. Referring to these figures, the tip 102 includes the body assembly 106 and the distally protruding tool 108. The body assembly 106 includes a nose 124, a locking ring 126, and a slide mechanism 128. The forceps jaws forming the distal tip 112 extend through the protruding tool 108 and are assembled in the body assembly 106, and fixed in the body assembly 106 via the slide mechanism 128. FIG. 2 shows the tip 102 adjacent the engagement interface portion 120 of the main body 104. FIGS. 3A and 3B show the tip 102 attached to the engagement interface portion 120 of the main body 104, and FIGS. 4A and 4B shows only the body assembly 106 with the nose 124, the locking ring 126, and the slide mechanism 128.

The engagement interface portion 120 includes a distal end 130, an outer surface 132, an axis 134 (FIG. 2) and a lock feature, shown here as a radial groove 136. The radial groove 136, as the lock feature in the exemplary embodiment shown, includes a lock surface 138 transverse to the axis 134. Here, the lock surface 138 is the distal wall of the groove 136. The lock surface 138 is arranged to interface with a corresponding surface of the nose 124 to inhibit or prevent separation of the tip 102 from the main body 104 when the tip 102 is in a locked condition. While the lock feature is shown as the radial groove 136 on the outer surface 132, the lock feature may be any other structural feature or features configured to prevent removal of the tip 102 from the main body 104 via mechanical interference. The lock features may be, for example, a groove, a lip, a hole, a shoulder, or other feature. In some embodiments, the lock feature is disposed on an inner surface of the engagement interface portion 120.

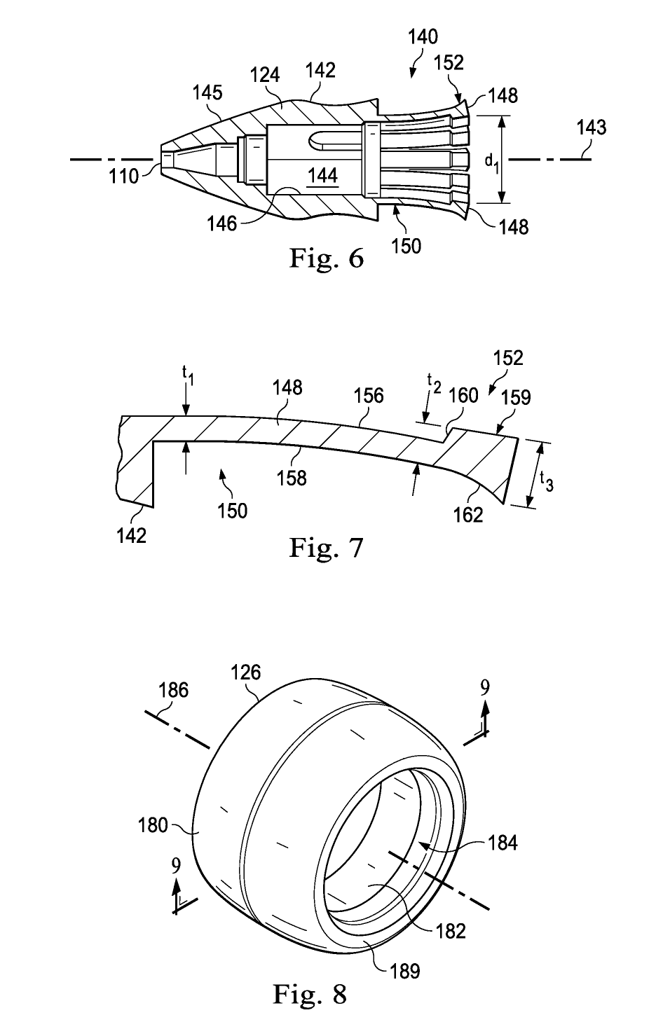

The following description is directed to individual components of the body assembly 106 and refers to FIGS. 2, 3A, 3B, 4A, and 4B, as well as additional drawings. FIGS. 5 and 6 show the nose 124 separate from the other components. The nose 124 includes the distal tip 110, a proximal end 140, and a tip body 142 having a longitudinal axis 143. A hollow passage 144 extends from the distal tip 110, through the tip body 142, and to the proximal end 140. The diameter of the hollow passage 144 varies in order to accommodate the protruding tool 108 and other components. The protruding tool 108 protrudes from the distal tip 110. In some embodiments, it is glued, welded or otherwise maintained in the distal tip.

As best seen in FIGS. 5 and 6, the tip body 142 has an outer surface 145 and an inner surface 146. A recess 147 is formed in the outer surface 145. An aperture 149 connects with the recess 147 and passes from the recess 147 to the inner surface 146. The recess 147 and the aperture 149 together are sized and shaped to receive the slide mechanism 128, as can be seen in FIGS. 4A and 4B. In the exemplary embodiment described, the tip body 142 includes two opposing recesses and apertures, spaced apart 180 degrees.

Referring to FIGS. 5 and 6, the proximal end 140 includes a plurality of flexible claws 148 configured to flex to engage and disengage with the engagement interface portion 120 of the main body 104. Each flexible claw 148 includes a distal portion 150 and a proximal end 152, and extends proximally from the tip body 142 of the nose 124. Each flexible claw 148 is configured to flex relative to the tip body 142 so that the proximal end 152 moves laterally toward or away from the longitudinal axis 143. In this example, the flexible claws 148 are monolithically formed with the tip body 142 so that the tip body 142 and the flexible claws 148 are formed of a single unitary structure.

FIG. 7 shows one exemplary flexible claw 148 connected to and extending from a portion of the tip body 142 of the nose 124. The flexible claw 148 includes an inner facing surface 156 and an outer facing surface 158. The distal portion 150 of the flexible claw 148 has a relatively smaller thickness t1 measured from the inner facing surface 156 to the outer facing surface 158, enabling each claw 148 to elastically deflect. The relatively smaller thickness t1 may continue from the distal portion 150 toward the proximal end 152 of the claw 148. The proximal end 152 includes a portion having a greater thickness t2 configured to mechanically engage a portion of the main body 104 when the tip 102 is connected to the main body 104. As can be seen in FIG. 7, the difference between the thickness t1 and the thickness t2 forms a protruding locking element 159 with an engagement surface 160 as a shoulder configured to mechanically engage with the engagement interface portion 120, such as the lock surface 138 forming a part of the radial groove 136. Thus, the proximal end 152 has a greater thickness t2 than the thickness of the extending portion of the flexible claw 148. Also, the outer facing surface 158 includes a slide stop shown as a curved engagement surface 162 configured to interface with the locking ring 126 as will be discussed below. Because of the curved engagement surface 162, the proximal end 152 has a thickness t3 that is greater than the thickness t2. The thickness t3, combined with the main body 104 when the main body 104 is attached to the tip 102, may mechanically inhibit or prevent the locking ring 126 from sliding in the proximal direction beyond the proximal end 152 of the flexible claws 148.

FIGS. 5 and 6 show the flexible claws 148 in a neutral or unloaded condition. The flexible claws 148 are biased with a curved condition where the proximal ends 152 together form an inner diameter d1 (FIG. 6) greater than an outer diameter d2 (FIG. 2) of the interface portion 120 of the main body 104. This enables the flexible claws 148 to not only deflect so that the protruding locking element 159 moves into engagement with the lock surface 138 of the engagement interface portion 120, but also enables the flexible claws 148 to disengage from the lock surface 138 of the engagement interface portion 120.

Although described with the flexible claws 148 engaging the lock surface 138 of the engagement interface portion 120 of the main body 104, other embodiments are arranged so that the flexible claws engage an inner surface of the interface portion of the main body 104 to selectively connect or disconnect the nose 124, and the rest of the body assembly 106, from the main body 104.

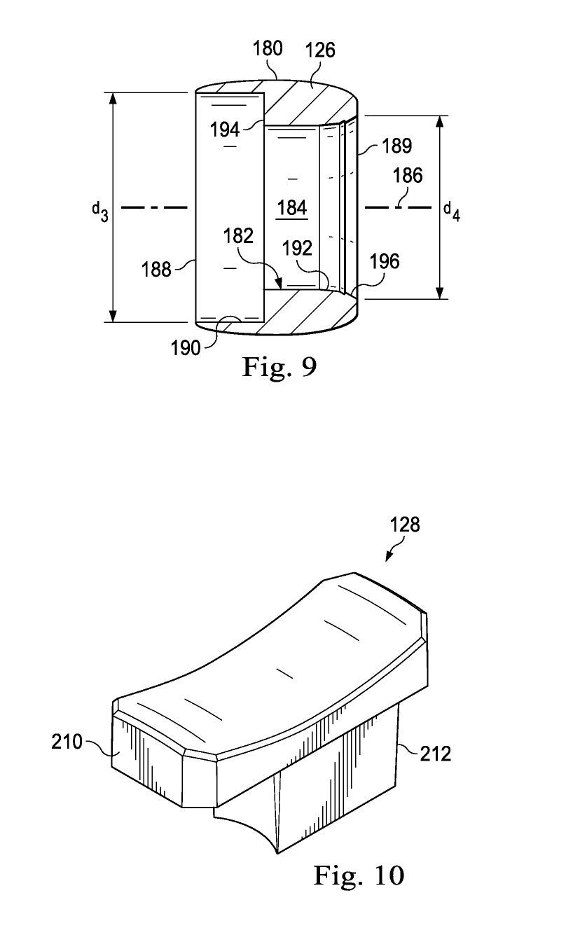

The locking ring 126 shown in FIGS. 8 and 9 is configured to slide axially relative to the nose 124 and cooperate with the nose 124 to attach to and detach the tip 102 from the main body 104. The locking ring 126 is shaped as a collar and includes an outer surface 180 and an inner surface 182 defining a passage 184 therethrough along a longitudinal axis 186. The locking ring 126 also includes a distal end 188 and a locking end 189.

The inner surface 182 includes a plurality of different levels or steps so that different regions of the inner surface have different diameters. For example, a first region 190 of the inner surface 182 of the locking ring 126 has a diameter d3, and a second region 192 of the inner surface 182 includes a diameter d4. A transverse stop 194 extends between the first and second regions 190, 192. The first region 190 is sized to receive a portion of the tip body 142 of the nose 124 when the locking ring 126 is in a distal position, which corresponds to an unlocked position. The transverse stop 194 mechanically limits the movement of the locking ring 126 in the distal direction by interfering with the proximal portion of the tip body 142.

The distal end 188 and the locking end 189 connect the inner and outer surfaces 182, 184. The locking end 189 is shaped with a curve 196 forming a part of the inner surface 182. The curve 196 is configured to interface with, at least in the embodiment shown, the slide stop or curved engagement surface 162 of the flexible claws 148 when the locking ring 126 is in the proximal or locked position. As such, in some embodiments, the curved engagement surface 162 and the curve 196 have about the same radius. The outer diameter of a circle formed by the curved engagement surface 162, when supported by the main body 104, has an inner diameter d4 greater than the diameter of the second region 192 of the locking ring 126. Accordingly, the curved engagement surface 162 acts as a mechanical stop to prevent the locking ring 126 from passing beyond and off of the flexible claws 148.

As can be seen in FIGS. 4A and 4B, the locking ring 126 fits about the flexible claws 148 and may be axially displaced relative to the flexible claws 148 to move the flexible claws 148 from an unlocked position to a locked position. Referring to FIG. 4B, when the locking ring 126 is in a distal position or unlocked position, it surrounds only the distal portion 150 of the flexible claws 148, and the proximal end 152 of the flexible claws 148 bend to their natural state. When in this natural state, in at least some embodiments, the engagement surface interface 120 of the main body 104 may be introduced into the hollow passage 144 formed in the nose 124. When the locking ring 126 is in a proximal position or locked position as shown in FIG. 4A, the second region 192 of the inner surface 182 interfaces with the flexible claws 148 to deflect the flexible claws 148 toward the longitudinal axis 143 of the nose 124. This brings the flexible claws 148 into engagement with the main body 104. For example, the proximal end 152 of the flexible claws 148 may project into the radial groove 138 as a recess or an annular depression, and may be disposed behind the lock surface 138, such as a ridge or other surface feature to mechanically interfere with the main body and prevent the main body from being released from the tip 102.

As can be seen in FIGS. 3A and 3B, the tip 102 includes a tool actuation mechanism 141 that is configured to cooperate with the actuation assembly 105 (FIG. 1) on the main body 104 to operate the tool 108. In the example shown, where the tool 108 is a forceps, the tool actuation mechanism 141 opens and closes the forceps jaws.

The tool actuation mechanism 141 may include, among other features, a biasing element, shown as a spring 170, an engager first portion 172, a pin 174, and an engager second portion 176. The engager first portion 172 and the engager second portion 176 may together form a single component with the pin 174 extending therethrough. The engager 172, 176 may axially displace within the body assembly 106. The spring 170 may bias the engager 172, 176 to a proximal position, resulting in opened forceps jaws.

In some embodiments, the tool actuation mechanism 141 actuates an instrument sleeve connected to the engager 172, 174 and forming a part of the tool 108. The actuation of the instrument sleeve may be in response to a central axial load applied against the engager 172, 176 by a cylindrical plunger (not shown) of the actuation assembly 105 on the main body 104.

The plunger may travel axially in the engagement surface interface 120 and may interface with and apply axial loading on the engager 172, 176. The plunger may displace the engager 172, 176 in the distal direction, against the biasing force of the spring 170. In some embodiments, this moves the instrument sleeve axially in the distal direction. As the instrument sleeve moves distally, the instrument sleeve closes the forceps jaws. For example, in some embodiments, the forceps jaws may be formed in a naturally open state and movement of the instrument sleeve relative to the jaws in the distal direction may result in the jaws being forced closed by the lumen of the instrument sleeve. As another example, the forceps jaws may be formed in a naturally closed state and movement of the instrument sleeve relative to the forceps jaws in the distal direction may result in the jaws opening or moving apart. This may be done using a dividing element associated with the instrument sleeve and disposed between the forceps jaws, for example.

Release of the central axial load permits the tool actuation mechanism 141 to automatically retract to its biased state, using the spring 170. This may result in the instrument sleeve moving proximally and allowing the forceps jaws to open. For example, as the instrument sleeve retracts from the naturally open forceps jaws, the jaws may open or separate. As another example, movement of the instrument sleeve proximally away from the naturally closed forceps jaws may allow the jaws to return to their naturally closed state.

Because of this simple actuation with an axial moving plunger, and without hooks or connectors between actuators, the tip 102 can be placed on the main body 104 without a particular alignment requirement. Accordingly, the connection with the tip and the main body is solely through the flexible claws 148, while the tool actuation mechanism 141 and the actuation assembly 105 interface only by contact, but not with a mechanical connection. This lack of mechanical connection between the tool actuation mechanism 141 and the actuation assembly 105 enables the tip 102 to be symmetrically connected to the main body 104 such that it is rotatable relative to the main body 104 about the axis when the tip 102 is in the locked condition. In some aspects, the actuation assembly 105 actuates the tool actuation mechanism 141 with a driver extending through the engagement interface portion.

FIG. 10 shows the slide mechanism 128 in greater detail. The slide mechanism 128 is configured to secure the tool actuation mechanism 141 in the tip body 142, and in some embodiments, does not have a direct role in the connection between the tip 102 and the main body 104. The slide mechanism 128 includes a protruding body 210 and an interface portion 212. The protruding body is sized to fit within the recess 147 and aperture 149 in the tip body 142 and interface with the operating mechanism disposed within the tip 102. Accordingly, the slide mechanism 128 may be used to secure the operating mechanism within the tip 102 so that the tip 102 is fully assembled and self-contained prior to being attached to the main body 104.

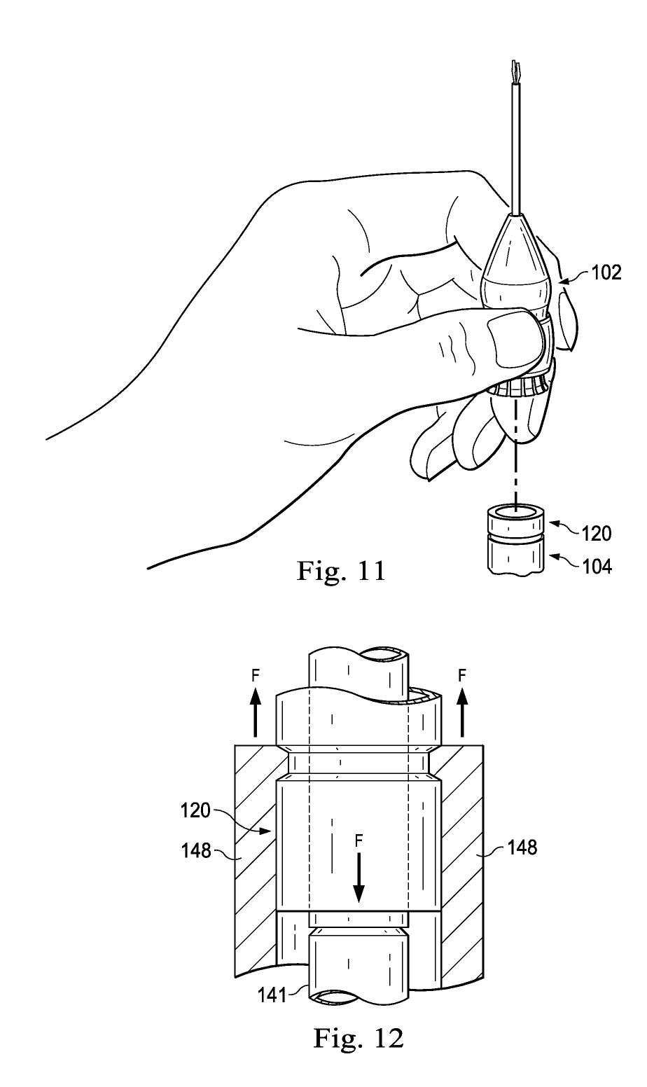

A method of attaching the tip 102 to the main body 104 will now be described with reference to FIG. 11. A user, such as a health care provider, may first confirm that the tip 102 is prepared for attachment to the main body 104. This may be done by observing whether the locking ring 126 is disposed at the proximal end 152 or the distal portion 150 of the flexible claws 148. If the locking ring 126 is disposed at the proximal end 152, the flexible claws 148 may be flexed inwardly in a manner that prevents introducing of the main body 104 to the tip 102. In such a case, the user may axially displace the locking ring 126 in the distal direction toward the proximal end 152. This permits the flexible claws 148 to elastically return to their neutral condition, so that, at least in the exemplary embodiment shown, they form the inner diameter d1 (FIG. 6) greater than the outer diameter d2 (FIG. 2) of the engagement surface interface 120 of the main body 104. Although described in terms of diameters of openings, other embodiments include polygonal shaped openings that may have widths instead of circular diameters. Yet other embodiments include flexible claws having a neutral position in the locked position. In such embodiments, the locking ring 126 may be used to displace the flexible claws to an unlocked position to release the main body 104.

With the flexible claws 148 in the neutral position, which in this embodiment is an open or unlocked position, the user may introduce the main body 104 into the passage 144 in the manner shown in FIG. 11. The user may do this by grasping the tip 102 and holding the locking ring 126 between fingers. Some embodiments require aligning certain features so that the main body, with any mechanisms carried by the main body 104, cooperatively aligns with any features carried by the tip 102. For example, this may include aligning mechanisms on the body 104 with actuation elements for forceps forming a portion of the tip 102.

With the engagement surface interface 120 of the main body 104 disposed between the flexible claws 148, the user may continue to advance the tip 102 onto the main body 104 until the end 130 of the engagement surface interface 120 abuts the interior of the tip body 142. At this point, continued advancement of the user's hand in the proximal direction moves only the locking ring 126. That is, the locking ring 126 then advances in the proximal direction relative to the flexible claws 148. As the locking ring 126 moves proximally, it forces the flexible claws 148 against their bias toward the main body 104. The diameter formed at the proximal ends 152 of the flexible claws 148 continues to decrease until the proximal ends 152 are mechanically disposed proximal of and directly in-line with physical structure of the main body 104. In the embodiment disclosed, this means the protruding locking elements 159 are disposed within an annular groove 136 disposed in the outer surface 132 of the engagement interfacing structure 120 of the main body 104. However, in other embodiments, the protruding locking element 159 may engage other features on the main body 104, and in some embodiments, may be disposed in an inner surface of the main body 104.

The locking ring 126 may advance until the locking end 189 of the locking ring 126, or more particularly, the curve 196 on the inner surface 182 of the locking ring 126, comes into contact with the curved engagement surface 162. The curved engagement surface 162 acts as a limit or stop that prevents the locking ring 126 from sliding off the flexible claws altogether. Thus, with the arrangement disclosed herein, a user can easily connect the tip 102 to the main body 104 quickly and easily using only one hand.

Removal of the tip is performed in a manner opposite the steps for attachment. Again, this may be performed with one hand, easily and simply. The user grasps the locking ring 126 between fingers, and the tip 102 is removed by sliding the locking ring 126 in the distal direction. This frees the flexible claws to return to their natural state, which is bent outwardly to form a diameter larger than the diameter of the engagement interfacing portion 120 of the main body 104. Accordingly, as the locking ring 126 moves distally, the proximal ends 152 of the flexible claws 148 radially move away from the central longitudinal axis 143, until they are out of the axial path of the engagement interfacing portion 120 of the main body 104. For example, the flexible claws may radially flex until they are no longer engaged in the radial groove 136 of the main body 104. Further movement in the distal directions separates the tip 102 from the main body 104. Depending upon the embodiment, the tip 102 may be discarded while the main body 104 may be sterilized through a chemical treatment, an autoclave treatment, or both.

FIG. 12 shows a stylized partial cross-sectional image of the main body 104 and flexible claws 148 in the locked position. Since the flexible claw arrangement extends symmetrically about the main body 104, lateral movement of the tip 102 is reduced or prevented, even at the distal tip 112 of the protruding tool 108. As shown in FIG. 12, the engaged flexible claws provide an equal and symmetrical force to counter the central force toward the distal end. Thus, the systems and methods disclosed herein may be more repeatable and predictable than prior devices that may have unequal or non-symmetric attachments. In preferred embodiments, the tool actuation mechanism 141 is actuated only by a central axial force from the main body 104.

Persons of ordinary skill in the art will appreciate that the embodiments encompassed by the present disclosure are not limited to the particular exemplary embodiments described above. In that regard, although illustrative embodiments have been shown and described, a wide range of modification, change, combination, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A surgical instrument for treating a medical condition of a patient, comprising:
a protruding tool configured to interface with and treat patient tissue;
a body assembly carrying the protruding tool, the body assembly comprising a plurality of flexible claws and a locking ring moveable along the flexible claws between a locked position and an unlocked position; and
a main body forming a hand grip portion to be grasped by a user during a medical procedure, the main body including an engagement interface portion configured to cooperate with the flexible claws to secure the body assembly to the main body when the locking ring is in the locked position.

2. The surgical instrument of claim 1, wherein the body assembly comprises a tip body and wherein each flexible claw of the plurality of flexible claws comprises a first portion attached to the tip body and a second portion opposite the first portion, the second portion comprising a protruding locking element configured to engage the engagement interface portion.

3. The surgical instrument of claim 2, wherein the protruding locking element comprises a shoulder surface that engages the engagement interface portion when the locking ring is in the locked position.

4. The surgical instrument of claim 2, wherein the first portion has a first thickness and the second portion has a second thickness greater than the first thickness, the first portion being arranged to elastically deflect under loading from the locking ring.

5. The surgical instrument of claim 2, wherein each flexible claw of the plurality of flexible claws comprises an inner facing surface and an outer facing surface, the inner facing surface comprising the protruding locking element, the outer facing surface comprising a slide stop that interfaces with the locking ring.

6. The surgical instrument of claim 1, wherein the plurality of flexible claws are biased to flare radially outwardly when the locking ring is in the unlocked position.

7. The surgical instrument of claim 1, wherein the flexible claws are symmetrically disposed in a radial configuration to provide symmetrical loading under an axial load.

8. The surgical instrument of claim 1, wherein the flexible claws and the engagement interface portion are shaped in a manner to permit rotation of the body assembly about the engagement interface portion when the locking ring is in the locked condition.

9. The surgical instrument of claim 8, wherein the body assembly carries a tool actuation mechanism, the tool actuation mechanism being actuatable via axial movement of an actuation assembly in the main body.

10. The surgical instrument of claim 1, wherein the engagement interface portion of the main body includes a radial groove that receives at least a portion of the flexible claws when the locking ring is in the locked position.

11. A surgical instrument for treating a medical condition, comprising:
a protruding, actuatable tool configured to interface with and treat patient tissue;
a body assembly having a distal end and a proximal end and a longitudinal axis extending therebetween, the tool projecting from the distal end along the longitudinal axis, the proximal end comprising a plurality of flexible claws symmetrically disposed about the longitudinal axis, and a locking element moveable axially between a locked position and an unlocked position;
a main body forming a gripping portion to be grasped by a user during a medical procedure, the main body comprising an axially aligned engagement interface portion configured to be engaged by the plurality of symmetrically disposed flexible claws so that axial loading on the engagement portion is countered by the flexible claws in a symmetrical manner.

12. The surgical instrument of claim 11, wherein the body assembly comprises a tip body and wherein each flexible claw of the plurality of flexible claws comprises a first portion attached to the tip body and a second portion opposite the first portion, the second portion comprising a protruding locking element configured to engage the engagement interface portion.

13. The surgical instrument of claim 12, wherein the protruding locking element comprises a shoulder surface configured to engage the engagement interface portion.

14. The surgical instrument of claim 12, wherein the first portion has a first thickness and the second portion has a second thickness greater than the first thickness, the first portion being arranged to elastically deflect under loading from the locking element.

15. The surgical instrument of claim 12, wherein each flexible claw of the plurality of flexible claws comprises an inner facing surface and an outer facing surface, the inner facing surface comprising the protruding locking element, the outer facing surface comprising a slide stop that interfaces with the locking element.

16. The surgical instrument of claim 11, wherein the plurality of flexible claws are biased to flare radially outwardly when the locking element is in the unlocked position.

17. The surgical instrument of claim 11, wherein the engagement interface portion of the main body includes a transverse lock surface that engages at least a portion of the flexible claws when the locking element is in the locked position.

18. A method of assembling a surgical instrument for treating a medical condition, comprising:
orienting a locking element on surgical tip relative to a plurality of flexible claws on the surgical tip so that the flexible claws are in an unlocked position;
introducing an engagement interface portion of a main body in an axial direction until the engagement interface portion is adjacent opposing flexible claws of the plurality of flexible claws, the engagement interface portion including a transverse lock surface; and
advancing the locking element, relative to the plurality of flexible claws and relative to the engagement interface portion, from an unlocked state to a locked state so that the flexible claws move from the unlocked position to a locked position where a portion of each flexible claw of the plurality of flexible claws is disposed axially in-line with the transverse locking surface to mechanically prevent removal of the engagement interface portion from the surgical tip.

19. The method of claim 18, wherein introducing an engagement interface portion until the engagement interface portion is adjacent opposing flexible claws of the plurality of flexible claws comprises introducing the engagement interface portion between opposing flexible claws.

20. The method of claim 18, wherein advancing the locking element from an unlocked state to a locked state so that the flexible claws move from the unlocked position to a locked position comprises deflecting the flexible claws radially inwardly.
